(19) United States
(12) Patent Application Publication
(10) Pub. No.: US 2024/0197708 A1
(43) Pub. Date: Jun. 20, 2024

(54) TREATMENT OF PRODROMAL HUNTINGTON DISEASE

(71) Applicant: Prilenia Neurotherapeutics Ltd., Yakum (IL)

(72) Inventors: Michael HAYDEN, Yakum (IL); Michal GEVA, Even- Yehuda (IL)

(73) Assignee: Prilenia Neurotherapeutics Ltd., Yakum (IL)

(21) Appl. No.: 18/553,758

(22) PCT Filed: Apr. 14, 2022
(86) PCT No.: PCT/IL2022/050396

§ 371 Date: Oct. 3, 2023

Related U.S. Application Data
(60) Provisional application No. 63/174,573, filed on Apr. 14, 2021.

(51) Int. Cl. A61K 31/451; A61P 25/14; A61P 25/28

(52) U.S. Cl. CPC A61K 31/451; A61P 25/14; A61P 25/28

(57) ABSTRACT

Disclosed herein are methods treating prodromal HD in a subject, wherein the subject has at least 36 CAG repeats in the huntingtin (Htt) gene, wherein the method comprises administering a composition comprising pridopidine or a pharmaceutically acceptable salt thereof.

### FIELD OF THE INVENTION

This invention provides methods of treating prodromal Huntington disease in a subject, wherein the subject has at least 36 CAG repeats in the huntingtin (Htt) gene, wherein the method comprises administering a composition comprising pridopidine or a pharmaceutically acceptable salt thereof.

### BACKGROUND OF THE INVENTION

### Huntington Disease

Huntington disease (HD) is a rare, fatal neurodegenerative disorder with an autosomal dominant mode of inheritance. The disease is characterized by progressive motor abnormalities, cognitive decline, psychiatric and behavioral symptoms. The signs and symptoms of HD develop gradually over many years, commonly leading to a diagnosis based on characteristic motor symptoms (‘motor onset’) between the ages of 30 and 50. With disease progression, patients experience functional decline, increasing disability, loss of independence, and premature death within 15-30 years of symptom onset. This gradual deterioration is evidence of the neurodegenerative processes occurring throughout the patient's lifetime.

HD is caused by an expansion in the number of CAG repeats in Exon 1 of the huntingtin (Htt) gene. Subjects with CAG repeat length of 35 or lower (non-carriers) are not at risk for developing HD. CAG repeat length of 36 and above are associated with disease. Higher repeat length is associated with earlier disease onset and more severe disease progression

The widespread neurodegeneration preceding the onset of HD in gene carriers can be monitored using specific imaging measures in the brain, and by measuring levels of biofluid biomarkers. A decrease in brain volume can be seen through the brain, and especially in the striatal substructures the caudate and putamen.

HD is divided into premanifest and manifest stages.

Subjects carrying the gene expansion for HD (≥36 CAG repeat length) who do not meet the criteria for clinical diagnosis of disease are referred to as “premanifest”. Premanifest HD subjects have no clinical diagnosis, as these subjects do not yet manifest sufficient motor symptoms to make the diagnosis.

The premanifest stage of HD is divided into two separate and distinct stages: the presymptomatic stage and the prodromal stage. A subject at the presymptomatic stage is clinically indistinguishable from a healthy, aged-matched individual and does not demonstrate any features of HD.

A prodromal subject is distinct from presymptomatic and demonstrates subtle motor, cognitive or psychiatric signs or features indicative of detectable neuroanatomical changes (i.e. a decrease in the volume of the brain and its substructures caudate and putamen). These features are not observed in a presymptomatic subject (FIG. 1). However, prodromal subjects maintain complete functional capacity.

Manifest HD patients are distinct from prodromal subjects (FIG. 2). Manifest HD is formally diagnosed on the basis of motor signs which are a defining feature of HD. Chorea is the most evident motor symptom, which is noticeable in manifest patients, but not observed in prodromal subjects. Additionally, HD manifest patients show impairments in other motor features including hyperkinesia or bradykinesia, and impairment in gross motor coordination skills, speech difficulties, gait, and postural deficits. Manifest HD patients also show significant decline in cognitive function and functional capacity. (FIG. 2).

### Currently Available Treatments

There are only two approved drugs for the treatment of chorea in manifest HD, tetrabenazine (Xenazine) and deutetrabenazine (Austedo). The two drugs share a common mechanism of action, blocking VMAT2 (vesicular monoamine transporter 2) uptake of dopamine into synaptic vesicles, interrupting the transport and degradation of dopamine. Importantly, these drugs can only treat chorea once it manifests, and cannot prevent the appearance of chorea. There are currently no approved drugs or treatments for prodromal subjects that can improve symptoms or delay or prevent the manifestation of HD.

Applicants have shown that pridopidine maintains, improves or lessens the decline of functional capacity in early-stage HD (early-stage HD refers to a manifest patient in its early stage of HD), as disclosed in U.S. Pat. Nos. 10,322,119 and 11,207,310. As mentioned above, prodromal subjects do not demonstrate a decline in functional capacity, which is evident in manifest HD patients.

There is a significant unmet need to develop medications to delay manifestation of HD or improve, maintain or lessen the decline of symptoms in the prodromal stage, to delay the onset of the disease. The prodromal stage can last many years with features which are different from, and do not lead to the diagnosis of manifest HD.

### Pridopidine

Pridopidine (4-[3-(methylsulfonyl)phenyl]-1-propyl-piperidine) (formerly known as ACR16) is a selective and high affinity sigma-1 receptor (SIR) agonist (Ki=0.57 μM).

### SUMMARY OF THE INVENTION

In some embodiments, the present disclosure provides a method of treating a prodromal Huntington disease in a subject who has at least 36 CAG repeats in the huntingtin (HTT) gene, wherein said method comprises administering a composition comprising pridopidine or a pharmaceutically acceptable salt thereof.

In some embodiments, the present disclosure provides a method of treating a prodromal Huntington disease in a subject, wherein the subject has at least 36 CAG repeats in the huntingtin (HTT) gene, wherein said method comprises administering a composition comprising pridopidine or a pharmaceutically acceptable salt thereof, wherein said prodromal subject has Unified Huntington's Disease Rating Scale-Total Functional Capacity (UHDRS-TFC) of 13. In other embodiments, said prodromal subject has a Diagnostic Confidence Level (DCL) of 1, 2 or 3 and not 4 (which confirms the diagnosis of HD). In other embodiments, said prodromal subject is characterized by impairment of motor functions comprising Total Motor Scale (TMS) with a score of between 5 and 10. In other embodiments, said prodromal subject has an Independence Score (IS) ≥90%. In another embodiment, the prodromal subject is Stage 1 or Stage 2 in the HD Integrated Staging System (HD-ISS).

In one embodiment, the composition for use in the methods of this invention comprises pridopidine or a pharmaceutically acceptable salt thereof and Compound 1:

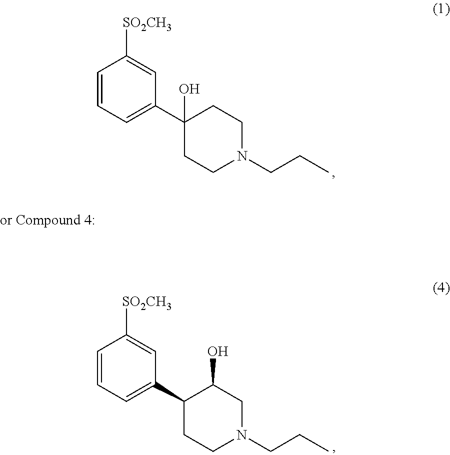 [SMILES: CCCN1CCC(O)(c2cccc(S(C)(=O)=O)c2)CC1 ; CCCN1CC[C@@H](c2cccc(S(C)(=O)=O)c2)[C@@H](O)C1]

or combination thereof; or pharmaceutically acceptable salts thereof.

In one embodiment, the method comprises administering pridopidine or a pharmaceutically acceptable salt between 10 mg/day-225 mg/day.

In one embodiment, the composition comprises Compound 1, Compound 4, combination thereof or pharmaceutically acceptable salt thereof, wherein Compound 1 or Compound 4 have a weight percentage of 0.001%-1.0% relative to pridopidine.

### BRIEF DESCRIPTION OF THE FIGURES

The subject matter regarded as the invention is particularly pointed out and distinctly claimed in the concluding portion of the specification. However, the invention both as to organization and method of operation, together with objects, features, and advantages thereof, may best be understood by reference to the following detailed description when read with the accompanying drawings in which:

FIG. 1: a table demonstrating the clinical differences between Prodromal HD subjects and presymptomatic HD gene carriers. Whole brain volume is expressed as percent of total intracranial volume and loss vs. age-matched controls or presymptomatic subjects as disclosed. Caudate and putamen volumes are divided by intra-cranial volume and multiplied by 1000. The antisaccade error test evaluates the ability to inhibit a reflexive response (reflexive inhibition), higher scores indicate more impairment. Ocular movement is measured using the United Huntington Disease Rating Scale-Total Motor Score (UHDRS-TMS) subitems for ocular movement. Stroop word reading (SWR) test examines the ability to inhibit cognitive interference, lower scores indicate more impairment. The trail making test A evaluates decision making and visual attention by measuring the time it takes to connect a set of numbered dots, higher scores indicate more impairment. In the semantic fluency task, a participant is asked to list as many objects from a given category within 1 minute, lower scores indicate more impairment. In the phonemic fluency task, participants are asked to list as many words as possible that start and end with specific letters, lower scores indicate more impairment. In the non-verbal fluency task participants are asked to draw as many drawings as possible within 5 minutes, lower scores indicate more impairment. In the spot the change test the number correct (k) corrected to the total number of guesses is demonstrated. Lower values indicate worsening. k=(H+CR−1)N, where H=#hits, CR=#correct rejections, N=#items displayed=5. The Symptom Checklist-90-Revised instrument helps evaluate a broad range of psychological problems and symptoms of psychopathology. BDI—Beck Depression Inventory—a widely-used, self-reporting questionnaire that assesses severity of depression, higher scores indicate more severe depression. BHS—Beck Hopelessness Scale—measures three major aspects of hopelessness: feelings about the future, loss of motivation, and expectations, and can be used as an indicator of suicidality; higher scores indicates a more severe condition.

FIG. 2: a table presenting the clinical differences between Prodromal HD subjects and manifest HD patients. Whole brain volume is expressed as percent of total intracranial volume and loss vs. age-matched controls or presymptomatic subjects as disclosed. The antisaccade error test evaluates the ability to inhibit a reflexive response (reflexive inhibition), higher scores indicate more impairment. The speeded tapping test evaluates precision in fine motor function, higher variability indicates more impairment, SD—standard deviation. Tongue protrusion force is measured using a precalibrated force transducer, the variability is expressed as a log coefficient of variance. Stroop word reading (SWR) test examines the ability to inhibit cognitive interference, lower scores indicate more impairment. The symbol digits modalities test (SDMT) examines concentration and decision making, lower scores indicate more impairment. The trail making test evaluates decision making and visual attention by measuring the time it takes to connect a set of dots (with either numbers (A) or a mix of numbers and letters (B)), higher scores indicate more impairment. The information sampling (beads task) assesses how much information patients gather before making a decision by counting the number of beads drawn. UPSIT—University of Pennsylvania Smell Identification Test—a common, reliable test to identify scents, lower scores indicate more impairment. In the emotion recognition task, a participant is asked to identify emotions from pictures of human faces, and the number of correct answers is evaluated; lower scores indicate greater impairment. The Visual Object and Space Perception (VOSP) battery of tasks assesses object recognition and perceptual processing. A participant is asked to identify silhouettes of 15 animals or objects, and the number of correct answers counted. Lower scores indicate more impairment. In the spot the change test the number correct (k) corrected to the total number of guesses is demonstrated. Lower values indicate worsening. k=(H+CR−1)N, where H=#hits, CR=#correct rejections, N=#items displayed=5.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention.

In some embodiments, provided herein, a method of treating prodromal Huntington disease in a subject who has at least 36 CAG repeats in the huntingtin (HTT) gene, wherein said method comprises administering a composition comprising pridopidine or a pharmaceutically acceptable salt thereof.

In some embodiments, provided herein, a method of treating prodromal Huntington disease in a subject who has at least 36 CAG repeats in the huntingtin (HTT) gene, wherein said method comprises administering a composition comprising pridopidine or a pharmaceutically acceptable salt thereof and at least one compound of compounds 1-7 or a pharmaceutically acceptable salt thereof; wherein compounds 1-7 are represented by the following structures:

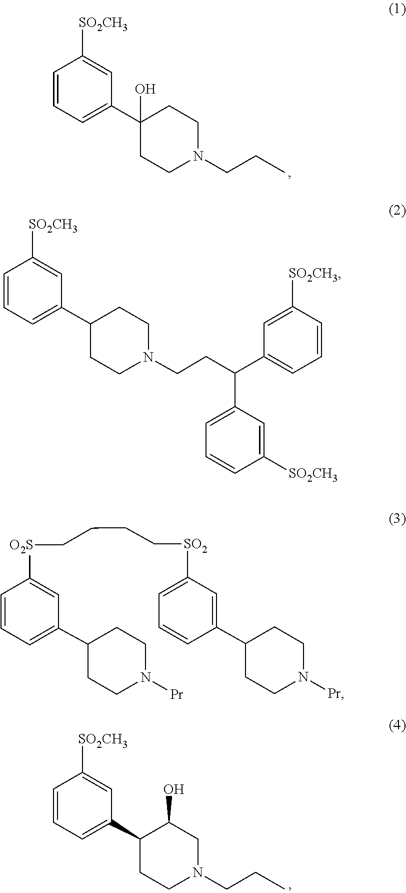

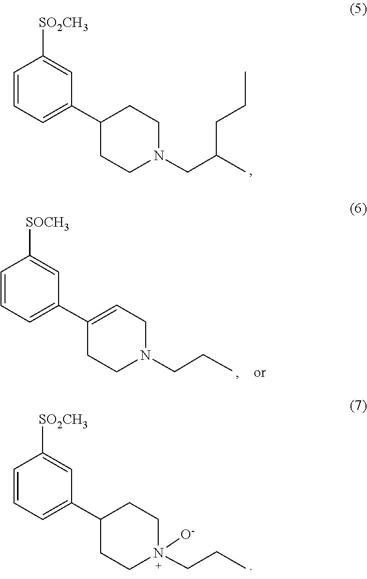

In some embodiments, provided herein, a method of treating prodromal Huntington disease in a subject, wherein the subject has at least 36 CAG repeats in the huntingtin (HTT) gene, wherein said method comprises administering a composition comprising pridopidine or a pharmaceutically acceptable salt thereof and Compound 1, Compound 4, combination thereof; or pharmaceutically acceptable salt thereof.

In some embodiments of the methods, compositions and uses disclosed herein the pharmaceutically acceptable salt of pridopidine, Compound 1, or Compound 4 is selected from the group consisting of: hydrochloride, hydrobromide, hydroiodide, nitrate, sulfate, bisulfate, phosphate, acid phosphate, isonicotinate, acetate, lactate, salicylate, citrate, D,L-tartrate, L-tartarate, D-tartarate, pantothenate, bitartrate, ascorbate, succinate, hemisuccinate, maleate, gentisinate, gentisate, fumarate, gluconate, glucaronate, glycolate, saccharate, formate, besylate, benzoate, glutamate, malate, methanesulfonate, ethanesulfonate, benzensulfonate, p-toluenesulfonate, oxalate, tosylate, naphtalen-2-sulfate, or pamoate (i.e., 1,1′-methylene-bis-(2-hydroxy-3-naphthoate)) salts. perchlorateaconate,cinnamate's citrate, embonate, enantate, malonate, mandelate, phthalate, sorbate, stearate salt. In other embodiment, the pharmaceutically acceptable salt is the hydrochloride salt. In another embodiment, the composition comprises pridopidine hydrochloride salt.

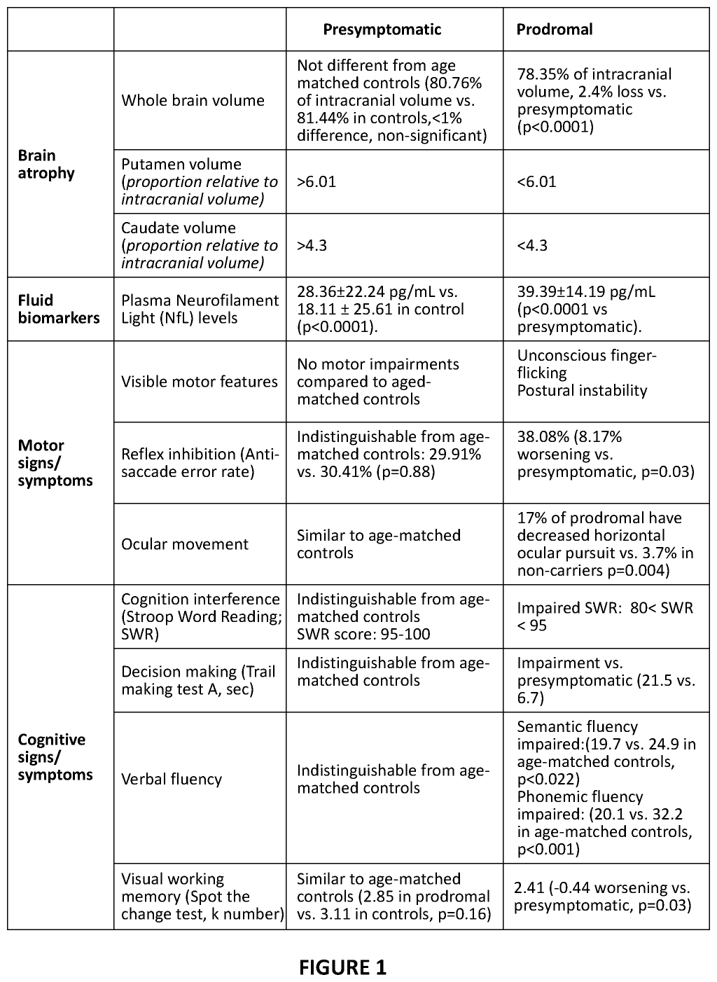

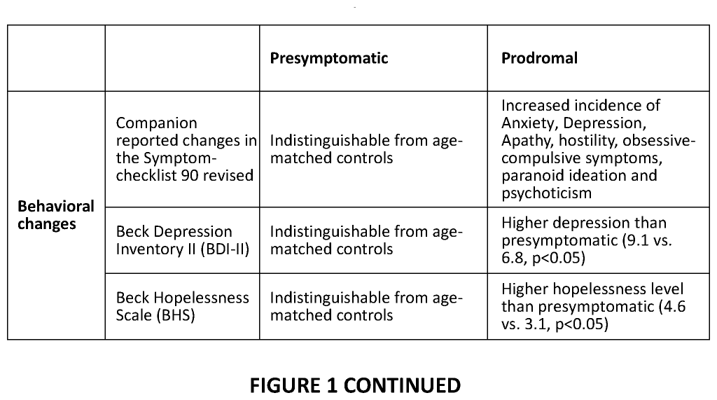

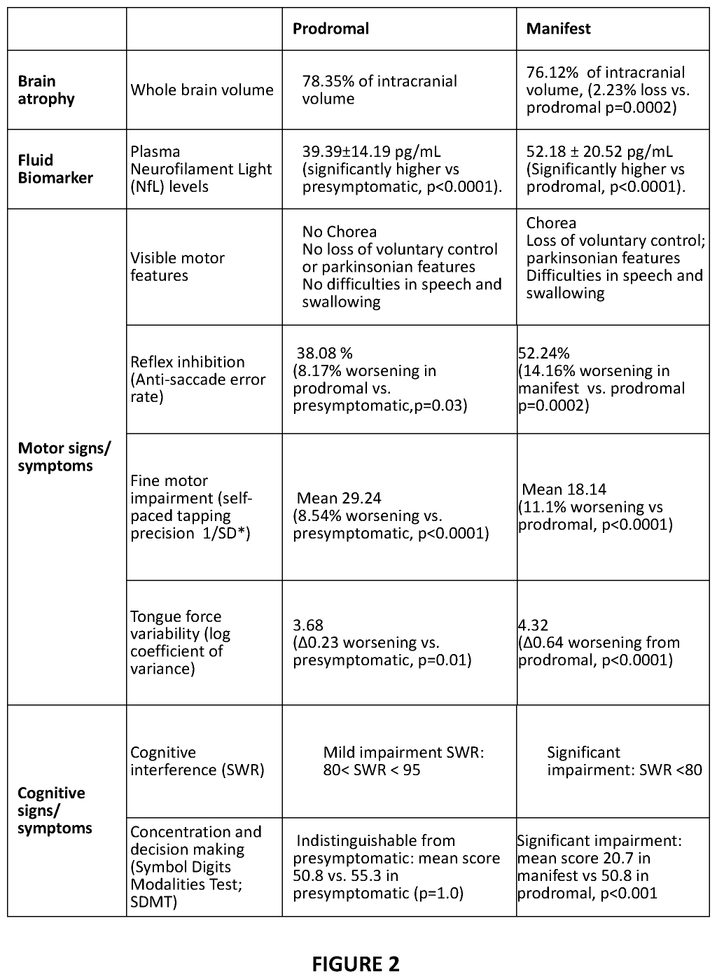

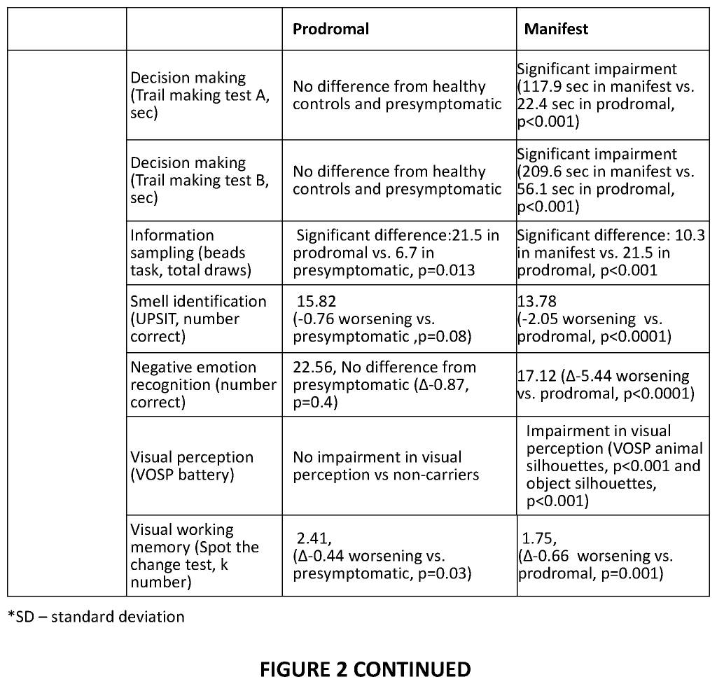

A skilled artisan would understand that a prodromal HD subject refers to a subject with at least 36 CAG repeats in the huntingtin (HTT) gene and has features that differentiate from presymptomatic subjects as defined in FIG. 1. A prodromal subject lacks the signs and symptoms required for a clinical diagnosis of manifest HD.

### Diagnosis and Clinical Assessment of HD Stages

A number of clinical and diagnostic measures are used to assess the signs and symptoms of HD. These measures differentiate between presymptomatic, prodromal and manifest HD stages. For example, the Unified Huntington Disease Rating Scale (UHDRS) assesses motor, functional, cognitive and behavioral domains. The diagnostic confidence score or level (DCL) relates to the level of confidence that motor impairments result from HD.

### DCL

In another embodiment, a prodromal HD subject has a Diagnostic Confidence Level (DCL) of 1, 2 or 3.

Clinical assessment of prodromal subjects includes a diagnostic confidence score or level (DCL), which evaluates the clinician's belief that the motor signs represent HD from 0 (no motor abnormalities) to 4 (motor abnormalities ≥99% likely to be due to HD). At DCL stage 0 a subject has no motor disabilities, hence is considered presymptomatic. DCL stages 1, 2 and 3 are considered prodromal. At DCL stage 1 motor abnormalities are considered by the clinician to be non-specific to HD (less than 50% confidence). At DCL stage 2, prodromal, motor abnormalities may be signs of HD (50-89% confidence). At DCL stage 3 motor abnormalities are likely to be a result of HD with 90-98% confidence, and at this stage cognitive and behavioral signs may also be evident, leading to a diagnosis of manifest HD. At DCL stage 4 a clinician is 99% or more confident that the motor signs and symptoms are a result of HD, and a formal diagnosis of HD can be made without additional cognitive and behavioral deficits. This model is advantageous as motor symptoms are relatively robust and easily identifiable amid the heterogeneity of the disease.

In one embodiment, the invention provides a method of treating prodromal HD in a subject with at least 36 CAG repeats in the huntingtin gene such that a patient maintains DCL of 1-3 over a period of at least six months, at least one year, at least 2 years, at least 3 years, at least 4 years, at least 5 years, at least 6 years, at least 7 years, at least 8 years, at least 9 years or at least 10 years. In another embodiment, a treated subject maintains DCL stage 1-3 for a period of over 10 years. In another embodiment, the DCL of the treated subject is decreased by 1 increment. In another embodiment the DCL of the treated subject is deceased by 2 increments. In another embodiment the DCL of the treated subject is decreased by 3 increments. In another embodiment, the DCL of the treated subject is decreased to 0.

### Disease Burden Score (DBS) CAG-Age Product (CAP) Score

The age of clinical onset of manifest HD is variable, and is influenced by the length of the CAG repeat expansion in the HTT gene. Similarly, CAG repeat length affects the rate of disease progression. The disease burden score (DBS) and the CAG-age product (CAP) score are both calculated as a function of age and CAG repeat length using the following equation: DBS or CAP=age×(CAG-L). L is a constant value in the range of 30-35 that anchors CAG length approximately at the lower end of the distribution relevant to HD pathology. Commonly used values are 35.5 and 33.66. In some instances, CAP score is normalized.

This score provides an index of the length and severity of the individual's exposure to the toxic effects of the mutant HTT gene. The DBS or CAP score are used to convey longitudinal data from cohorts of patients with a range of ages and CAG repeat lengths.

Several studies report DBS or CAP scores in participants. The Cambridge cohort study reported the DBS score of a small cohort of presymptomatic (n=9) and prodromal subjects (n=10) to be 181.5±38.1 and 296.1±60.9, respectively (Mason et al., Predicting clinical diagnosis in Huntington's disease: An imaging polymarker. Ann Neurol. 2018 Mar.; 83(3):532-543). In the TRACK-HD study the DBS scores for presymptomatic (n=60) and prodromal subjects (n=58) were 237.9±31.4 and 312.8±32.5, respectively (Tabrizi et al., Biological and clinical manifestations of Huntington's disease in the longitudinal TRACK-HD study: cross-sectional analysis of baseline data. Lancet Neurol. 2009 Sep.; 8(9):791-801). DBS or CAP score at conversion to manifest HD usually exceeds 400 (Ross et al., Huntington disease: natural history, biomarkers and prospects for therapeutics. Nat Rev Neurol. 2014 April; 10(4):204-16. doi: 10.1038/nrneurol.2014.24. Epub 2014 Mar. 11).

The recently created HD Integrated Staging System (ISS) defines specific stages based on distinct clinical landmarks and differentiates between presymptomatic, prodromal and manifest HD patients.

### HD Integrated Scoring System (HD-ISS)

In another embodiment, a prodromal HD subject is characterized by an ISS stage of 1 or 2.

The HD-ISS is an evidence-based staging system that addresses all stages of HD. The ISS defines landmark assessments and cut-off values to identify critical transitions in disease stages. HD-ISS has 4 distinct stages. ISS Stage 0 (presymptomatic) includes all HD gene carriers for whom there is no detectable change in pathological markers, signs or symptoms related to HD. In ISS stage 1 (prodromal), pathological changes indicating neurodegeneration and an onset of specific signs can be detected, specifically changes to the volume of the caudate and putamen brain substructures. ISS Stage 2 (prodromal) is defined by the presence of definite clinical signs and symptoms, both cognitive and motor without any decline in functional capacity. ISS Stage 3 (manifest) is accompanied by a decline in functional capacity.

Table 1 summarizes the differences between presymptomatic, prodromal and manifest stages using the Disease Burden Score (DBS) or CAG-age product (CAP) score, the DCL and the HD Integrated Staging System (HD-ISS).

TABLE 1
Different clinical diagnostics of presymptomatic,
prodromal and manifest HD stages.

| | Presymptomatic | Prodromal | Manifest |
|---|---|---|---|
| Disease Burden | <250 | 250-400 | >400 |
| Score (DBS)/ | | | |
| CAP Score* | | | |
| Motor DCL | 0 | 1-3 | 4 |
| HD-ISS | Stage 0 | Stages 1 + 2 | Stage 3 |

*CAP: CAG-Age product, CAP = age × (CAG-L). L is a constant value in the range of 30-35 that anchors CAG length approximately at the lower end of the distribution relevant to HD pathology. DBS - = age × (CAG-35.5)

Additionally, there are some clinical scales which are unique to the prodromal stage, i.e. the scale developed by the Functional Rating Scale Taskforce for pre-Huntington Disease (FuRST-pHD) specifically to assess changes in prodromal HD subjects.

### Clinical Tools Specific for Evaluating Disease Progression in Prodromal HD

### Functional Rating Scale Taskforce for Pre-Huntington Disease (FuRST-pHD)

FuRST-pHD is a scale based on patient-reported outcomes that is sensitive to changes in premanifest HD. FuRST is designed to be sensitive to changes specifically in prodromal HD such as work ability, social interaction and financial transactions. Seven interview questions discriminate between prodromal and manifest early HD. These included balance on one foot (0.51 in prodromal vs. 1.56 in manifest), balance when walking (0.44 vs. 1.17), fine motor ability (0.16 vs. 1.28), complex motor behavior (0.18 vs. 0.9), writing (0.49 vs. 1.55), clumsiness (0.55 vs. 1.29) and functional impact (0.44 vs. 1.24) (Vaccarino et al., Assessment of motor symptoms and functional impact in prodromal and early huntington disease. PLOS Curr. 2011 Jun. 14; 2:RRN1244).

### Independence Score (IS)

The UHDRS-IS comprises part of the UHDRS functional assessments (Huntington's Study Group 1996). It is a rating scale where the patient's degree of independence is given in percentage, from 10% (tube fed, total bed care) to 100% (no special care needed). Scores must end in 0 or 5 (eg, 10%, 15%, 20% etc).

In one embodiment, a prodromal HD subject has an IS ≥90%. In another embodiment, a prodromal HD subject has an IS ≥95%. In another embodiment, the prodromal HD subject has an IS equal to 95%. In another embodiment, the prodromal subject has an IS equal to 100%.

In some embodiments, provided herein, a method of treating prodromal Huntington disease in a subject, wherein the subject has at least 36 CAG repeats in the huntingtin (HTT) gene, wherein said method comprises administering a composition comprising pridopidine or a pharmaceutically acceptable salt thereof, as measured by maintaining or improving or slowing the decline in change from baseline in the UHDRS-Independence Scale (UHDRS-IS) in a prodromal HD subject.

In one embodiment, a subject treated with the composition comprising pridopidine demonstrates an improvement of 5% in IS. In another embodiment, a subject treated with the composition comprising pridopidine maintains the same IS for a period of 6 months. In another embodiment, a subject treated with the composition comprising pridopidine maintains the same IS for a period of 1 year, 2 years, 3 years, 4 years, 5 years, 6 years, 7 years, 8 years, 9 years or 10 years. In another embodiment, a subject treated with the composition comprising pridopidine maintains the same IS for over 10 years.

In one embodiment, a subject treated with the composition comprising pridopidine demonstrates a worsening in IS that is 5% less than the worsening seen in an untreated subject over the same period of time. By maintaining IS, the diagnosis of manifest HD can be delayed.

### Motor Function

Prodromal HD subjects have subtle motor behavioral and cognitive features that differentiate them from presymptomatic subjects, but lack the definitive motor symptoms necessary for clinical diagnosis of manifest HD.

Motor function is most commonly assessed by the Total Motor Score of the UHDRS (UHDRS-TMS).

### TMS

In another embodiment, a prodromal HD subject is characterized by impairment of motor function comprising Total Motor Scale (TMS) with a score of between 5 and 10. In another embodiment, the prodromal subject is characterized by impairment of motor function comprising Total Motor Scale (TMS) with a score of 10. In another embodiment, with a score of 9. In another embodiment, with a score of 8. In another embodiment, with a score of 7. In another embodiment, with a score of 6. In another embodiment, with a score of 5.

In another embodiment, a prodromal subject is characterized by unconscious finger-flicking, mild impairment and decreased horizontal ocular pursuit (Wild, E. J. and S. J. Tabrizi (2014). Huntington's Disease. Premanifest and Early Huntington's Disease, Oxford University Press).

In some embodiments, provided herein a method of treating prodromal Huntington disease in a subject, wherein the subject has at least 36 CAG repeats in the huntingtin (HTT) gene, wherein said method comprises administering a composition comprising pridopidine or a pharmaceutically acceptable salt thereof, as measured by maintaining or improving or slowing the decline of motor function in a prodromal HD subject.

The motor ability may be measured, for example, by the UHDRS Total Motor Score (TMS) score, the UHDRS TMS score excluding chorea or UHDRS TMS score excluding dystonia, or the modified Motor Score (mMS) which excludes both chorea and dystonia. In the TMS scale, lower values indicate better motor function. Hence, a decrease in TMS indicates an improvement in motor function.

In one embodiment, a prodromal subject treated with the composition comprising pridopidine demonstrates an improvement in the UHDRS-TMS of at least 1 unit, at least 2 units, least 3 units, at least 4 units, or at least 5 units. In another embodiment, a prodromal subject treated with the composition comprising pridopidine demonstrates an improvement of between 5 to 10 units in the TMS.

In one embodiment, a prodromal subject treated with the composition comprising pridopidine maintains TMS for a period of 6 months, 1 year, 2 years, 3 years, 4 years, 5 years, 6 years, 7 years, 8 years, 9 years or 10 years. In another embodiment, a prodromal subject treated with the composition comprising pridopidine maintains TMS for a period of 10-15 years. In another embodiment, a prodromal subject treated with the composition comprising pridopidine maintains TMS for over 15 years.

In one embodiment, a prodromal subject treated with the composition comprising pridopidine demonstrates a decline in TMS that is at least 1 unit less than the decline demonstrated by an untreated matched subject over the same period of time. In one embodiment a prodromal subject treated with the composition comprising pridopidine demonstrates a worsening in TMS that is 5-10 units less than the worsening of an untreated matched subject over the same period of time. In some embodiments, a prodromal subject treated with the composition comprising pridopidine demonstrates a worsening in TMS that is more than 10 points less than the worsening observed in an untreated subject over the same period of time.

In one embodiment, a prodromal subject treated with the composition comprising pridopidine demonstrates a maintenance of variability in GAITrite stride length for a period of 6 months, 1 year, 2 years, 3 years, 4 years, 5 years, 6 years, 7 years, 8 years, 9 years or 10 years. In another embodiment a prodromal subject treated with the composition comprising pridopidine maintains the variability in GAITrite stride length for a period of 10-15 years. In another embodiment, a prodromal subject treated with the composition comprising pridopidine maintains the variability in GAITrite stride length for over 15 years.

In one embodiment, a prodromal subject treated with the composition comprising pridopidine demonstrates a reduction in the variability of GAITrite stride length of at least 1%, at least 5%, at least 10% or at least 20%.

In one embodiment, a prodromal subject treated with the composition comprising pridopidine demonstrates a reduction in the variability of GAITrite stride length that is at least 1% less than the worsening demonstrated by an untreated subject over the same period of time. In one embodiment, a prodromal subject treated with the composition comprising pridopidine demonstrates a worsening in GAITrite that is 5-10% less than the decline of an untreated subject over the same period of time. In some embodiments, a prodromal subject treated with the composition comprising pridopidine demonstrates a worsening in GAITrite that is more than 10% less than the worsening observed in an untreated subject over the same period of time.

In some embodiments, provided herein, a method of treating prodromal Huntington disease in a subject, wherein the subject has at least 36 CAG repeats in the huntingtin (Htt) gene, wherein said method comprises administering a composition comprising pridopidine or a pharmaceutically acceptable salt thereof as measured by maintaining or improving or slowing the decline in change from baseline in the gait and balance score as defined by the sum of the UHDRS-Total Motor Score (UHDRS-TMS) domains gait, tandem walking and retropulsion pull test in a prodromal HD subject.

Prodromal HD subjects additionally demonstrate changes in quantitative motor assessments as detailed below.

### Quantitative-Motor (Q-Motor)

Q-Motor is an objective assessment of specific motor functions that utilizes pre-calibrated and temperature-independent force transducers and three-dimensional position sensors to provide standardized, unbiased measurements. Q-Motor has been used to detect motor signs in manifest, prodromal, and premanifest HD cohorts. Q-Motor is a sensitive measure that correlates with changes in brain volume, and Total Functional Capacity (TFC) and TMS sections of the UHDRS.

In another embodiment a prodromal HD subject has decreased Q-Motor finger tap speed frequency, decreased Q-Motor finger tap Inter-Onset-Interval, decreased Q-Motor finger tap Inter-Tap-Interval, decreased Q-Motor inter-peak interval, decreased Q-Motor pronate/supinate hand tapping frequency, decreased Q-Motor hand tapping inter-onset interval, decreased grip force, decreased tongue force or any combination thereof compared to healthy controls.

A quantifiable motor assessment that differentiates prodromal from presymptomatic subjects and from manifest HD patients is speeded finger tapping. In this assay, participants are required to tap with their index finger on a tapping apparatus at the highest possible velocity between two auditory cues. The variability of tap duration, inter-onset, inter-tap and inter-peak intervals are measured. In the TRACK-HD study, prodromal subjects demonstrate significant differences from presymptomatic subjects in all of these measures (all p<0.05). Similarly, prodromal subjects demonstrated significant differences from manifest HD patients (all p<0.0001) (Bechtel et al., Tapping linked to function and structure in premanifest and symptomatic Huntington disease. Neurology. 2010 Dec. 14; 75(24):2150-60).

In another embodiment, a prodromal HD subject is characterized by a significant reduction in Tongue force variability compared to healthy controls.

In one embodiment, a prodromal subject treated with the composition comprising pridopidine shows no worsening in the Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval, decreased grip force, tongue force or any combination thereof over 1 year, 2 years, 3 years, 4 years, 5 years, 6 years, 7 years, 8 years, 9 years or 10 years. In another embodiment, a prodromal subject treated with the composition comprising pridopidine shows an improvement of at least 1% in the Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval, decreased grip force, tongue force or any combination thereof. In other embodiments, a prodromal subject treated with the composition comprising pridopidine shows an improvement of at least 2%, at least 3%, at least 4%, at least 5% or 10%.

In one embodiment, by treating the features described herein in a prodromal subject, onset of symptoms of manifest HD is delayed by at least 6 months, 1 year, 2 years, 3 years, 4 years, 5 years, 6 years, 7 years, 8 years, 9 years or 10 years. In another embodiment, by treating the symptom described herein, manifest HD onset is delayed by 10-15 years. In another embodiment, by treating the symptom described herein, onset of manifest HD is delayed by over 15 years.

In some embodiment, the method provided herein comprises improving impairments of motor functions as measured by Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval or any combination thereof.

In one embodiment, a prodromal subject treated with the composition comprising pridopidine demonstrates an improvement in Q-Motor measurements of at least 1%, at least 5%, at least 10%, or at least 15%. In another embodiment, a prodromal subject treated with a composition comprising pridopidine demonstrates an improvement of between 15 to 25% in Q-Motor measurements.

In one embodiment, a prodromal subject treated with the composition comprising pridopidine maintains/shows no decline in Q-Motor measurements of Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval or any combination thereof for a period of 6 months, 1 year, 2 years, 3 years, 4 years, 5 years, 6 years, 7 years, 8 years, 9 years or 10 years. In another embodiment, a prodromal subject treated with the composition comprising pridopidine maintains/shows no decline in Q-Motor measurements of Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval or any combination thereof for a period of 10-15 years. In another embodiment, a prodromal subject treated with a composition comprising pridopidine maintains/shows no decline in Q-Motor measurements of Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval or any combination thereof for over 15 years.

In one embodiment, a prodromal subject treated with the composition comprising pridopidine demonstrates less worsening in Q-Motor measurements of Q-Motor finger tap speed frequency, Q-Motor finger tap Inter-Onset-Interval, Q-Motor finger tap Inter-Tap-Interval, Q-Motor inter-peak interval, Q-Motor pronate/supinate hand tapping frequency, Q-Motor hand tapping inter-onset interval or any combination thereof that is at least 5% less than an untreated subject over the same period of time. In one embodiment, a prodromal subject treated with the composition comprising pridopidine demonstrates less worsening in Q-Motor measurements that is 5-20% less than the worsening in an untreated subject over the same period of time. In some embodiments, a prodromal subject treated with a composition comprising pridopidine demonstrates a worsening in Q-Motor measurements that is 20-50% less than the worsening observed in an untreated subject over the same period of time. In some embodiments, a prodromal subject treated with a composition comprising pridopidine demonstrates a worsening in Q-Motor measurements that is ≥50% less than the worsening observed in an untreated subject over the same period of time.

### Oculomotor Function

Prodromal HD subjects show a difference in antisaccade error rate compared to presymptomatic and manifest HD patients. In the antisaccade test, an individual focuses eye gaze on a motionless object, and a stimulus is presented to one side. The individual is asked to make a saccade (a quick, simultaneous movement of both eyes) in the direction opposite to a presented stimulus. An error occurs when the individual fails to inhibit the reflexive saccade towards the stimulus. The antisaccade test requires both willful motion and the ability to inhibit the reflexive response to look at the stimulus. Antisaccade errors are indicative of dysfunction of the brain substructures putamen, supplementary motor area and frontal eye field, Higher error rates indicate worsening.

In the TRACK-HD study, presymptomatic HD subjects do not differ from healthy controls in the antisaccade error rate (−0.5% p=0.88). However, prodromal HD subjects demonstrate a significantly increased error rate (worsening) compared to presymptomatic HD subjects (+8.17% in prodromal vs. presymptomatic, p=0.03). The antisaccade error rate also differentiates prodromal HD subjects from early manifest HD patients with manifest showing a significant increase (worsening) error rate compared to prodromal (+14.16% in manifest HD vs. prodromal, p=0.0002). Thus, prodromal subjects are distinct from both presymptomatic subjects and manifest HD patients in their antisaccade error rate (Tabrizi et al., Biological and clinical manifestations of Huntington's disease in the longitudinal TRACK-HD study: cross-sectional analysis of baseline data. Lancet Neurol. 2009 Sep.; 8(9):791-801).

In one embodiment, a prodromal subject treated with the composition comprising pridopidine demonstrates no worsening in antisaccade error rate over a period of at least 6 months, 1 year, 2 years, 3 years, 4 years, 5 years, 6 years, 7 years, 8 years, 9 years or 10 years. In another embodiment, the prodromal subject treated with a composition comprising pridopidine demonstrates no worsening in antisaccade error rate over a period of 5-15 years. In some embodiment, the prodromal subject treated with a composition comprising pridopidine demonstrates no worsening in antisaccade error rate for over 10 years.

In one embodiment, a prodromal subject treated with the composition comprising pridopidine demonstrates a decrease (has improvement) in the antisaccade error rate of at least 1%, at least 2%, at least 3%, at least 4% or at last 5%. In another embodiment, a prodromal subject treated with the composition comprising pridopidine demonstrates a decrease (improvement) in the antisaccade error rate of between 5 and 10%. In some embodiment, a prodromal subject treated with the composition comprising pridopidine demonstrates a decrease (improvement) in antisaccade error rate of more than 10%.

In one embodiment, a prodromal subject treated with the pharmaceutical composition comprising pridopidine demonstrates less worsening in the antisaccade error rate than an untreated prodromal subject by 1%, 2%, 3%, 4% or 5% over the same period of time. In another embodiment, a prodromal subject treated with a composition comprising pridopidine demonstrates less worsening in antisaccade error rate that is 5-10% smaller than an untreated prodromal subject over the same period of time. In some embodiment, the worsening observed in a prodromal subject treated with a composition comprising pridopidine is more than 10% smaller than the worsening observed in an untreated prodromal subject over the same period of time.

### Ocular Motion

Ocular abnormalities were studied in a cohort of prodromal (n=35) and presymptomatic (n=35) HD subjects and compared to non-carrier controls (n=27). Ocular pursuit was measured using the ocular pursuit items in the UHDRS-TMS scale, which are rated from 0 (normal) to 4 (cannot perform, most severe). Presymptomatic subjects and non-carrier controls showed similar frequency of affected horizontal ocular pursuit (5.7% vs 3.7%, respectively). On the other hand, a significantly greater percent of prodromal subjects demonstrated worsening in ocular pursuit (17.1%) compared to non-carrier controls, (p=0.004). Similarly, Vertical pursuit showed a similar frequency with 22.9% affected in the presymptomatic group, and 22.2% affected in the non-carrier group. Prodromal HD had a significantly higher frequency of affected subjects of 42. 9%, p=0.075 vs. non-carriers (Winder J Y, Roos R A C. Premanifest Huntington's disease: Examination of oculomotor abnormalities in clinical practice. PLOS One. 2018 Mar. 1; 13(3):e0193866).

### Cognition

Prodromal HD Subjects Differ from Presymptomatic Subjects and Manifest HD Patients in Cognitive Measures

The prodromal subject's cognitive domains may be measured, for example, by the cognitive assessment battery (CAB), which consists of the following tests: Symbol Digit Modalities Test; Paced Tapping; One Touch Stockings of Cambridge (abbreviated); Emotion Recognition; Trail Making B; Hopkins Verbal Learning Test.

In one embodiment, a prodromal HD subject is characterized by cognitive change assessed by the HD Cognitive Assessment Battery (CAB) of tests composite, Paced Tapping, One-Touch stockings of Cambridge, Emotion Recognition, Trail making test A and B, Hopkins Verbal learning test, Map Search, Smell Identification, Spot the Change, Judgement of Line Orientation, speeded tapping and Montreal Cognitive Assessment (MoCA) scores, in which the score indicates worsening compared to healthy controls.

The prodromal subject's cognitive domains may also be measured by the Hopkins Verbal Learning Test—Revised (HVLT-R). The prodromal subject's cognitive domains may additionally be measured by the Paced Tapping test, the Montreal Cognitive Assessment (MoCA) scale or the Symbol Digit Modalities Test (SDMT). The prodromal subject's cognitive domains may additionally be measured by trail making test B (TMT-B), Circle tracing-direct, Circle tracing-indirect or combination thereof.

Further provided is a method of improving, preventing the decline, or delaying cognitive decline of a prodromal HD subject.

In some embodiment, provided herein, a method of treating a prodromal subject who has at least 36 CAG repeats in the huntingtin (Htt) gene, wherein said method comprises administering a composition comprising pridopidine or a pharmaceutically acceptable salt thereof, wherein the method comprises improving, preventing the decline, or delaying cognitive decline of a prodromal HD subject.

### Symbol Digit Modalities Test (SDMT)

One of the most sensitive measures for assessing cognitive decline is the Symbol Digits Modalities test (SDMT). A participant is presented with a row of symbols corresponding to the digits 1-9, and is tasked with writing or orally reporting the correct digit in a row that contains only the symbols. The SDMT measures visuospatial attention, processing speed and working memory.

In multiple natural history studies looking at HD gene carriers before and after formal HD diagnosis, both baseline values and the longitudinal decline in SDMT differentiate between presymptomatic, prodromal and manifest HD patients.

Presymptomatic HD subjects in the HD-Young Adult Study (YAS) demonstrate similar SDMT values as non-HD carriers (59.7 vs. 60.7, p=0.62).

The difference in SDMT between prodromal subjects (n=16) and manifest HD patients (n=22) was evaluated in a study investigating cognitive flexibility. HD manifest patients scored significantly worse than prodromal subjects (20.7±10.4 vs. 50.8±8.2, p<0.0001, lower values indicate higher severity) (Heim et al., Time will tell: Decision making in premanifest and manifest Huntington's disease. Brain Behav. 2020 Nov.; 10(11):e01843).

Presymptomatic subjects do not differ from age-matched non-carrier controls in any neuropsychiatric or neurocognitive measures compared to non-carrier controls.

Longitudinal decline in the SDMT is a sensitive measure of cognitive decline. Prodromal subjects (n=46) demonstrate a significant annual change from baseline of −4.11 (CI 95% −6.73 to −1.49, p=0.003) compared to healthy controls. Presymptomatic subjects, (n=58) did not show any difference from healthy controls (p=0.346) (Tabrizi et al., Predictors of phenotypic progression and disease onset in premanifest and early-stage Huntington's disease in the TRACK-HD study: analysis of 36-month observational data. Lancet Neurol. 2013 Jul.; 12(7):637-49; Tabrizi et al., Potential endpoints for clinical trials in premanifest and early Huntington's disease in the TRACK-HD study: analysis of 24 month observational data. Lancet Neurol. 2012 January; 11(1):42-53).

In one embodiment, a prodromal HD subject is characterized by impairments of cognitive functions comprising Symbol Digit Modalities Test (SDMT) score being above 40; Stroop Word reading (SWR) score being above 80; annulus length in the circle tracing direct test greater than 6.6 (log cm); annulus length in the circle tracing indirect test greater than 5.4 (log cm); spot the change 5 sec difference number correct score (A) being reduced by −0.4 to −1.5 compared to healthy control; or any combination thereof.

The invention additionally provides a method of treating a prodromal subject who has at least 36 CAG repeats in the huntingtin (Htt) gene, wherein said method comprises administering a composition comprising pridopidine or a pharmaceutically acceptable salt thereof, as measured by maintaining or improving or slowing the decline in the SDMT test.

In one embodiment the prodromal subject administered a composition comprising pridopidine or a pharmaceutically acceptable salt thereof maintains SDMT score over 6 months, over 1 year, over 2 years, over 3 years, over 4 years, over 5 years, over 6 years, over 7 years, over 8 years, over 9 years or over 10 years. In another embodiment, a prodromal subject treated with the composition comprising pridopidine maintains SDMT score for over 10 years.

In one embodiment, a prodromal subject treated with the composition comprising pridopidine shows an improvement of 1-20 points in the SDMT score. In one embodiment the SDMT score of a prodromal subject treated with the composition comprising pridopidine is improved by up to 5 points. In another embodiment, the SDMT score of a prodromal subject treated with the composition comprising pridopidine is improved by 5-10 points. In some embodiment, the SDMT score is improved by 10-15 points. In other embodiments, the SDMT score is improved by 15-20 points. In some embodiment, the SDMT score of a prodromal subject treated with the composition comprising pridopidine is improved by 20-30 points. In some embodiment, the SDMT score is improved by 30-40 points.

In one embodiment a prodromal subject treated with the composition comprising pridopidine shows a slowing in the decline of SDMT score by at least 1 point per year compared to a non-treated subject. In another embodiment a prodromal subject treated with the composition comprising pridopidine shows a slowing in the decline of SDMT score by at least 2 points per year compared to a non-treated subject. In another embodiment a prodromal subject treated with the composition comprising pridopidine shows a slowing in the decline of SDMT score by at least 3 points per year compared to a non-treated subject. In one embodiment the prodromal subject treated with a composition comprising pridopidine shows a slowing in the decline of SDMT score by 1-10 points per year compared to a non-treated subject.

### Stroop Word Test

The invention additionally provides a method of treating a prodromal subject who has at least 36 CAG repeats in the huntingtin (Htt) gene, wherein said method comprises administering a composition comprising pridopidine or a pharmaceutically acceptable salt thereof, as measured by maintaining or improving or slowing the decline in change from baseline in the Stroop Word test. In one embodiment the prodromal subject treated with a composition comprising pridopidine maintains SWR score over 6 months, over 1 year, over 2 years, over 3 years, over 4 years, over 5 years, over 6 years, over 7 years, over 8 years, over 9 years or over 10 years. In another embodiment, the prodromal subject treated with a composition comprising pridopidine maintains SWR score for over 10 years.

In one embodiment, a prodromal subject treated with the composition comprising pridopidine shows an improvement of 1-20 points in the SWR score. In one embodiment the SWR score of a prodromal subject treated with the composition comprising pridopidine is improved by up to 5 points. In another embodiment, the SWR score of a prodromal subject treated with the composition comprising pridopidine is improved by 5-10 points. In some embodiment, the SWR score of a prodromal subject treated with the composition comprising pridopidine is improved by 10-points. In other embodiments, the SWR score of a prodromal subject treated with the composition comprising pridopidine is improved by 15-20 points. In some embodiment, the SWR score is improved by up to 20 points.

In one embodiment a prodromal subject treated with the composition comprising pridopidine shows a slowing in the decline of SWR score by at least 1 point per year compared to a non-treated subject. In another embodiment a prodromal subject treated with the composition comprising pridopidine shows a slowing in the decline of SWR score by at least 2 points per year compared to a non-treated subject. In another embodiment a prodromal subject treated with the composition comprising pridopidine shows a slowing in the decline of SWR score by at least 3 points per year compared to a non-treated subject. In one embodiment a prodromal subject treated with the composition comprising pridopidine shows a slowing in the decline of SWR score by 1-10 points per year compared to a non-treated subject.

### Verbal and Non-Verbal Fluency

Verbal and non-verbal fluency was studied in presymptomatic (n=16) and prodromal HD (n=16) subjects and compared to non-carrier controls (n=38) in a Swedish cohort. In the phonemic fluency test, participants were requested to produce as many words as possible starting with S and ending with A in a 3-minute time period, and the total number of words and errors was counted. In the semantic fluency test, participants were asked to say as many words as possible in two categories, fruits and vegetables over a 1-minute period. The number of words were counted. Non-verbal fluency was evaluated using a design fluency test. Participants were requested to draw as many unique drawings that cannot represent real objects as possible within 5 minutes. The number of drawings adhering to these rules were counted. Presymptomatic HD carriers show no difference from non-carrier controls in phonemic (27.6=8.9 vs. 32.2±9.6, p=0.322) and semantic fluency (24.2±5.5 vs. 24.9±7.0, p=1). On the other hand, prodromal subjects show significant worsening from non-carrier controls in phonemic fluency (20.1±10.2 vs, 32.2±9.6, p<0.001) and semantic fluency (19.7±4.9 vs. 24.9±7.0, p<0.022) (Robins Wahlin et al., Non-Verbal and Verbal Fluency in Prodromal Huntington's Disease. Dement Geriatr Cogn Dis Extra. 2015 Dec. 18; 5(3):517-29).

### Visual Perception

Visual perception deficits are present in manifest HD and can discriminate prodromal HD subjects from manifest HD patients. Visual perception was assessed in prodromal HD subjects (n=22), manifest HD patients (n=22) and non-carrier controls (n=18) using the animal silhouette test and the object silhouette test. The number of correct answers is counted; lower scores indicate worsening. Prodromal HD subjects did not show any difference from non-carrier controls in either test. However, prodromal HD subject differed significantly from manifest HD patients in both the animal silhouette test (12.2±1.6 vs. 9.2±1.9, p<0.001) and the object silhouette test (10.3±3.5 vs. 7.2±3.1, p=0.001) (Coppen et al., Visual Object Perception in Premanifest and Early Manifest Huntington's Disease. Arch Clin Neuropsychol. 2019 Nov. 27; 34(8): 1320-1328).

In some embodiments, provided herein a method of treating a prodromal HD subject who has at least 36 CAG repeats in the huntingtin (Htt) gene, wherein said method comprises administering a composition comprising pridopidine or a pharmaceutically acceptable salt thereof, as measured by composite Unified Huntington's Disease rating scale (cUHDRS), UHDRS clinical measures of TFC, TMS, SDMT, and SWR.

### Behavioral Abnormalities

In some embodiment, provided herein a method of treating a prodromal subject who has at least 36 CAG repeats in the huntingtin (Htt) gene, wherein said method comprises administering a composition comprising pridopidine or a pharmaceutically acceptable salt thereof, wherein the method comprises improving behavioral abnormalities in a prodromal HD subject. In one embodiment, the behavioral abnormalities comprise depressed mood, suicidal ideation, anxiety, irritability, angry or aggressive behavior, apathy, perseverative thinking or behavior, obsessive-compulsive behavior, paranoid thinking or delusions, hallucinations, disoriented behavior or any combination thereof.

In one embodiment, the Problem Behavior Assessment Score (PBA-S) comprises depressed mood, suicidal ideation, anxiety, irritability, angry or aggressive behavior, apathy, perseverative thinking or behavior, obsessive-compulsive behavior, paranoid thinking or delusions, hallucinations and disoriented behavior. In the PBA-s, higher scores indicate worsening.

In one embodiment, a prodromal HD subject has behavioral changes including irritability and apathy, comprising Problem Behaviors Assessment (PBA) apathy score difference compared to healthy control being between 0.5-1 or PBA irritability score compared to healthy control being between 1.3-1.8.

The prodromal subject's behavior and/or psychiatric state may also be measured by the Problem Behaviors Assessment for irritability. The prodromal subject's behavior and/or psychiatric state may also be measured by the Problem Behaviors Assessment for lack of initiative or apathy. The prodromal subject's behavior and/or psychiatric state may also be measured by the Problem Behaviors Assessment short form apathy sub-item. The prodromal subject's behavior and/or psychiatric state may also be measured by the Apathy Evaluation Scale (AES). The prodromal subject's behavior and/or psychiatric state may be measured by the Problem Behaviors Assessment for obsessive-compulsiveness. The prodromal subject's behavior and/or psychiatric state may also be measured by the Problem Behaviors Assessment for disoriented behavior. In some embodiments, the hum prodromal subject's behavior and/or psychiatric state is measured by the Problem Behaviors Assessment short form apathy sub-item or the Problem Behaviors Assessment-short form (PBA-S). The PBA-S evaluates the following items for both severity and frequency: depressed mood, suicidal ideation, anxiety, irritability, angry or aggressive behavior, apathy, perseverative thinking or behavior, obsessive-compulsive behavior, paranoid thinking or delusions, hallucinations, and disoriented behavior.

In one embodiment, a prodromal subject treated with a composition comprising pridopidine maintains total PBA-s score over 6 months, over 1 year, over 2 years, over 3 years, over 4 years, over 5 years, over 6 years, over 7 years, over 8 years, over 9 years or over 10 years. In another embodiment, the subject treated with a composition comprising pridopidine maintains PBA-s irritability score for over 10 years. In one embodiment, the prodromal subject treated with a composition comprising pridopidine maintains total PBA-s score over 6 months, over 1 year, over 2 years, over 3 years, over 4 years, over 5 years, over 6 years, over 7 years, over 8 years, over 9 years or over 10 years. In another embodiment, the prodromal subject treated with a composition comprising pridopidine maintains PBA-s irritability score for over 10 years.

In one embodiment, the prodromal subject treated with a composition comprising pridopidine shows an improvement of 0.5-10 units in the PBA-s score. In one embodiment the PBA-s score of a prodromal subject treated with a composition comprising pridopidine is improved by up to 1 unit. In another embodiment, the PBA-s score is improved by 1-5 units. In some embodiment, the PBA-s score of a prodromal subject treated with a composition comprising pridopidine is improved by 5-10 units.

In one embodiment the prodromal subject treated with a composition comprising pridopidine shows a slowing in the decline of PBA-s score by at least 0.5 units per year compared to a non-treated subject. In another embodiment the prodromal subject treated with a composition comprising pridopidine shows a slowing in the decline of PBA-s score by at least 1 unit per year compared to a non-treated subject.

Depression was compared in presymptomatic (n=207) and prodromal HD subjects (n=284) participating in the PREDICT-HD longitudinal observational trial. Depression was measured using the Beck Depression Inventory II (BDI-II) and the Beck Hopelessness Scale (BHS), both commonly used self-rated scales. Significant differences were observed between the two groups in both measures (p<0.05), indicating more severe depression and feelings of helplessness in prodromal HD subjects (Epping et al., Characterization of depression in prodromal Huntington disease in the neurobiological predictors of HD (PREDICT-HD) study. J Psychiatr Res. 2013 Oct.; 47(10):1423-31).

The invention further provides a method of preventing or delaying the decline in the behavior and/or psychiatric state of a prodromal HD subject.

In one embodiment, the prodromal subject treated with a composition comprising pridopidine maintains total BDI or BHS score over 6 months, over 1 year, over 2 years, over 3 years, over 4 years, over 5 years, over 6 years, over 7 years, over 8 years, over 9 years or over 10 years. In another embodiment, the prodromal subject treated with a composition comprising pridopidine maintains BDI or BHS score for over 10 years.

In one embodiment, the prodromal subject treated with a composition comprising pridopidine shows an improvement of 0.5-10 units in the BDI or BHS scores. In one embodiment the BDI or BHS score of a prodromal subject treated with a composition comprising pridopidine is improved by up to 1 unit. In another embodiment, the BDI or BHS score of a subject treated with a composition comprising pridopidine is improved by 1-5 units. In some embodiment, the BDI or BHS of a prodromal subject treated with a composition comprising pridopidine score is improved by 5-10 units.

In one embodiment the prodromal subject treated with a composition comprising pridopidine shows a slowing in the decline of BDI or BHS score by at least 0.5 units per year compared to a non-treated subject. In another embodiment a prodromal subject treated with a composition comprising pridopidine shows a slowing in the decline of BSI or BHS score by at least 1 unit per year compared to a non-treated subject.

Imaging and Fluid Biomarkers Differentiate Prodromal HD Subjects from Presymptomatic HD Gene Carriers and Manifest HD Patients

### Imaging Biomarkers

Prodromal subjects demonstrate unique imaging biomarkers which differentiate them from presymptomatic subjects and manifest HD patients, i.e. volumetric changes in the caudate and putamen brain substructures.

### Brain Volume

In one embodiment, a prodromal HD subject has neuroimaging abnormalities selected from: a decrease in total brain volume, a decrease in volume of the caudate, a decrease in the volume of the putamen, a decrease in white matter volume, a decrease in gray matter volume, an increase in ventricular volume or any combination thereof. In another embodiment, the prodromal subject has a decrease in total brain volume. In another embodiment, the prodromal subject has a decrease in volume of the caudate. In another embodiment, the prodromal subject has a decrease in volume of the putamen. In another embodiment, the prodromal subject has a decrease in white matter volume. In another embodiment, the prodromal subject has a decrease in gray matter volume.

In some embodiments, provided herein a method of treating a prodromal subject who has at least 36 CAG repeats in the huntingtin (Htt) gene, wherein said method comprises administering a composition comprising pridopidine or a pharmaceutically acceptable salt thereof is measured by maintaining or increasing or slowing the decrease in total brain volume, volume of the caudate, volume of the putamen, white matter volume, gray matter volume, or by maintaining or reducing or slowing the increase of ventricular volume. In one embodiment, the decrease in total brain volume is by about 1-4%. In another embodiment, the decrease in total brain volume is 2%. In one embodiment, the decrease in volume of the caudate is by about 10-25%. In one embodiment, the volume of the putamen is less than 4.3 when divided by the intracranial volume and multiplied by 1000. In one embodiment, the volume of the putamen is less than 6 when divided by the intracranial volume and multiplied by 1000. In one embodiment, the decrease in volume of the putamen is by about 10-25%. In one embodiment, the decrease in white matter volume is between 15-25 mL compared to healthy controls. In one embodiment, the decrease in gray matter volume is o between 15-25 mL compared to healthy controls. In one embodiment, the increase in ventricular volume is up to 25 mL compared to healthy controls.

### Functional Connectivity

Functional connectivity also differentiates prodromal HD subjects from presymptomatic subjects

The default-mode network (DMN) is a set of brain regions which are active when the brain is not engaged in a cognitive task, and which are deactivated upon cognitive engagement with a task. It is crucial for cognitive processing and can be assessed by resting-state fMRI (rs-fMRI).

Alterations in DMN function are associated with a decline of cognition in several neurodegenerative diseases, i.e. ALS, AD, and Frontotemporal Dementia (FTD). In HD, abnormal task-related brain activation patterns are observed early in prodromal HD subjects but not in presymptomatic subjects.

In one embodiment, a prodromal HD subject has a decrease in the default mode network (DMN) connectivity.

Functional connectivity in brain regions mapping to the DMN, the precuneous and inferior parietal lobe, was compared between far from onset (presymptomatic) and close to onset (prodromal) premanifest subjects. The groups were stratified by disease burden score (DBS). Median DBS differed significantly between the groups (224±44 in presymptomatic vs. 364±43 in prodromal, p=0.001). Prodromal HD subjects showed decreased left caudate nucleus functional connectivity in the posterior cingulate/precuneus (PCC, p=0.006) and in the inferior parietal lobe bilaterally (left, p=0.005; right, p=0.006). No significant differences were found between presymptomatic subjects and non-HD carriers (Pini et al., Striatal connectivity in pre-manifest Huntington's disease is differentially affected by disease burden. Eur J Neurol. 2020 Nov.; 27(11):2147-2157).

### Glucose Metabolism

Glucose Metabolism Differentiates Prodromal HD Subjects from Presymptomatic Subjects and Manifest HD Patients

In some embodiment, provided herein a method of treating prodromal HD in a subject who has at least 36 CAG repeats in the huntingtin (Htt) gene, wherein said method comprises administering a composition comprising pridopidine or a pharmaceutically acceptable salt thereof, wherein the method comprises enhancing uptake of glucose metabolism into the brain. In one embodiment, the brain region comprises the caudate, the putamen or any combination thereof. In another embodiment, the brain region comprises the caudate. In another embodiment, the brain region comprises the putamen. In another embodiment, the brain region comprises the caudate and the putamen.

Brain glucose metabolism, assessed by uptake of fluorodeoxyglucose (FDG) and measured by positron emission tomography ([18F]FDG-PET), is commonly used as a sensitive measure to assay disease progression. The relative FDG distribution is an indirect marker of regional synaptic activity, commonly used in the investigation of neurodegenerative diseases. In prodromal HD subjects, decreased metabolism is observed in the anterior striatum, caudate nucleus and putamen. No changes in glucose metabolism by FDG-PET are observed in presymptomatic subjects.

In a study in 12 prodromal HD subjects glucose metabolism was studied longitudinally over 44 months. At baseline, regional metabolism was reduced compared to control in the caudate and putamen (0.74±0.05 vs 0.93±0.02 in control, p<0.005) and in the cingulate cortex (0.88±0.01 vs 0.99±0.02 in control, p<0.0001). Regional metabolism progressively declined in the striatum (p<0.005) and in the thalamus (p<0.01)(Feigin et al., Thalamic metabolism and symptom onset in preclinical Huntington's disease. Brain. 2007 November; 130(Pt 11):2858-67. doi: 10.1093/brain/awm217. Epub 2007 Sep. 24)

Regional cerebral glucose utilization in the caudate was assessed in non-carrier controls (n=11), prodromal HD subjects (n=13) and manifest HD patients in early stages of the disease (n=10) using FDG-PET. Glucose utilization in prodromal subjects was slightly lower than that in non-carrier controls (7.2±1.08 mg/100 gm/min vs. 8.2±1.0 mg/100 gm/min, respectively), and significantly lower in HD patients (4.9±0.6 mg/100 gm/min, p<0.001) (Hayden et al., Positron emission tomography in the early diagnosis of Huntington's disease. Neurology. 1986 Jul.; 36(7):888-94).

Metabolic changes were assessed in presymptomatic subjects (n=8), prodromal subjects (n=7) and manifest HD patients (n=18) as well as 18 normal controls, using FDG-PET. In most of the presymptomatic subjects (7/8, 88%), FDG distribution in the striatum was normal. However, only 4/7 (57%) of the prodromal subjects had normal FDG distribution with 3/7, (43%) demonstrating mild hypometabolism. Manifest HD patients demonstrate significant hypometabolism when compared to both healthy controls (p<0.001) and prodromal subjects (p<0.001) (López-Mora et al., Striatal hypometabolism in premanifest and manifest Huntington's disease patients. Eur J Nucl Med Mol Imaging. 2016 Nov.; 43(12):2183-2189. doi: 10.1007/s00259-016-3445-y. Epub 2016 Jun. 28.)

In one embodiment, a prodromal HD subject is characterized by a decrease in uptake of glucose into the caudate nucleus; decrease in uptake of glucose into the pallidum; a decrease in uptake of glucose into the putamen compared to healthy controls; a decrease in the uptake of glucose into the striatum.

### Fluid Biomarkers

### Neurofilament Light Levels

Neurofilament light protein (NfL) levels in biofluids (i.e. cerebrospinal fluid (CSF), blood, serum and plasma) may be used as a biomarker of neurodegeneration in prodromal HD. Thus, NfL concentrations in plasma or serum or blood or CSF of HD prodromal subjects may provide a means for assessing and predicting neural damage in patients with prodromal HD. NfL levels in plasma and CSF were assessed in the TRACK-HD cohort. In non-carrier controls (n=97) plasma NfL levels averaged 18.11±25.61 pg/mL. In presymptomatic HD carriers (n=58) plasma NfL levels averaged 28.36±22.24 pg/mL (p<0.0001 vs control). In prodromal HD subjects (n=46) NfL levels averaged 39.39±14.19 pg/mL (p<0.0001 vs presymptomatic). In manifest HD patients in early stages of the disease (n=66) NfL levels averaged 52.18±20.52 pg/mL (p<0.0001 vs prodromal) (Byrne et al., Neurofilament light protein in blood as a potential biomarker of neurodegeneration in Huntington's disease: a retrospective cohort analysis. Lancet Neurol. 2017 August; 16(8):601-609).

In some embodiment, provided herein a method of treating a prodromal subject who has at least 36 CAG repeats in the huntingtin (Htt) gene, wherein said method comprises administering a composition comprising pridopidine or a pharmaceutically acceptable salt thereof, wherein the method comprises maintaining, preventing, or slowing the increase in Nfl. In another embodiment, the method comprises maintaining or reducing Nfl levels. In another embodiment, the method comprises preventing the increase in Nfl. In another embodiment, the method comprises slowing the increase in Nfl. In another embodiment, the method comprises reducing the levels of NfL.

In one embodiment, a prodromal HD subject is characterized by neurofilament (NfL) levels in the blood of 25-50 pg/mL and in the CSF up to 2000 pg/mL.

In another embodiment a prodromal HD subject has an increase in neurofilament light (NfL) levels in the plasma, serum, or cerebral spinal fluid (CSF).

In one embodiment, a prodromal subject treated with the composition comprising pridopidine maintains NfL levels for a period of 6 months, 1 year, 2 years, 3 years, 4 years, 5 years, 6 years, 7 years, 8 years, 9 years or 10 years. In another embodiment, a prodromal subject treated with the composition comprising pridopidine maintains NfL levels for a period of 10-15 years. In another embodiment, a prodromal subject treated with the composition comprising pridopidine maintains NfL levels for over 15 years.

In one embodiment, a prodromal subject treated with the composition comprising pridopidine demonstrates a reduction in the levels of plasma NfL of at least 1%, at least 2%, at least 3%, at least 4%, at least 5%, at least 6%, at least 7%, at least 8%, at least 9% or at least 10%. In another embodiment, a prodromal subject treated with the composition comprising pridopidine demonstrates a reduction of 10-20% in plasma NfL levels. In another embodiment, a prodromal subject treated with the composition comprising pridopidine demonstrates a reduction of 20-30% in plasma NfL levels. In another embodiment, a prodromal subject treated with the composition comprising pridopidine demonstrates a reduction of 30-40% in plasma NfL levels. In another embodiment, a prodromal subject treated with the composition comprising pridopidine demonstrates a reduction of 40-50% in plasma NfL levels.

### Neuroinflammation

In one embodiment, a prodromal HD subject is characterized by increased neuroinflammation, microglial activation, astrocytic activation, elevation of IL6 levels or any combination thereof.

In another embodiment, a prodromal HD subject has increased neuroinflammation, microglial activation, astrocytic activation or combination thereof. In another embodiment, the prodromal subject has increased neuroinflammation. In another embodiment, the prodromal subject has microglial activation. In another embodiment, the prodromal subject has astrocytic activation. In another embodiment, the prodromal subject has increased neuroinflammation and microglial activation and/or astrocytic activation.

CSF levels of the inflammatory biomarker interleukin-6 (IL-6) differentiate prodromal HD subjects from presymptomatic gene carriers

The immune system has been implicated in HD pathogenesis, and CSF levels of the pro-inflammatory cytokine IL-6 are elevated in HD. This elevation has been suggested to be an early occurrence that contributes to HD pathogenesis.

The HD-YAS study compared IL-6 levels in the CSF in presymptomatic HD gene carriers and non-carrier controls and found no difference between the two groups (0.98 vs. 1.01 log pg/mL in gene carriers and controls, respectively, p=0.68)

CSF levels of IL-6 were compared between non-carrier controls (n=14) and prodromal HD subjects (n=3,) IL-6 levels were significantly elevated in prodromal subjects vs. controls (0.9 vs. 0.7, log pg/mL p=0.041) (Scahill et al., Biological and clinical characteristics of gene carriers far from predicted onset in the Huntington's disease Young Adult Study (HD-YAS): a cross-sectional analysis. Lancet Neurol. 2020 June; 19(6):502-512)

In some embodiment, provided herein a method of treating a prodromal subject who has at least 36 CAG repeats in the huntingtin (Htt) gene, wherein said method comprises administering a composition comprising pridopidine or a pharmaceutically acceptable salt thereof, wherein the method comprises reducing neuroinflammation in a prodromal subject.

In one embodiment, a prodromal subject treated with the composition comprising pridopidine maintains IL-6 levels for a period of 6 months, 1 year, 2 years, 3 years, 4 years, 5 years, 6 years, 7 years, 8 years, 9 years or 10 years. In another embodiment, a prodromal subject treated with the composition comprising pridopidine maintains IL-6 levels for a period of 10-15 years. In another embodiment, a prodromal subject treated with the composition comprising pridopidine maintains IL-6 levels for over 15 years.

In some embodiment, a prodromal subject is differentiated from a presymptomatic gene carrier by increased levels of a fluid biomarker. In some embodiment, a prodromal subject is differentiated from a manifest HD patient by decreased levels of a biomarker.

In one embodiment, the biomarker is YKL-40 in the CSF. In one embodiment, the biomarker is IL-8 in the CSF or the plasma. In one embodiment, the biomarker is Neurogranin in the CSF. In one embodiment, the biomarker is tau in the CSF. In another embodiment, the biomarker is tau in the plasma. In one embodiment, the biomarker is phosphorylated tau in CSF. In another embodiment, the biomarker is phosphorylated tau in the plasma. In one embodiment, the biomarker is GFAP in CSF. In one embodiment, the biomarker is proenkephalin. In another embodiment, the biomarker is GFAP in plasma. In one embodiment the biomarker is total Htt protein. In another embodiment, the biomarker is mutant Htt (mHtt).

In one embodiment, a prodromal subject treated with the composition comprising pridopidine demonstrates a reduction in the levels of a fluid biomarker by at least 1%, at least 2%, at least 3%, at least 4%, at least 5%, at least 6%, at least 7%, at least 8%, at least 9% or at least 10%. In another embodiment, a prodromal subject treated with the composition comprising pridopidine demonstrates a reduction of 10-20% in fluid biomarker levels. In another embodiment, a prodromal subject treated with the composition comprising pridopidine demonstrates a reduction of 20-30% in fluid biomarker levels. In another embodiment a prodromal subject treated with the composition comprising pridopidine demonstrates a reduction of 30-40% in fluid biomarker levels. In another embodiment, a subject treated with the composition comprising pridopidine demonstrates a reduction of 40-50% in fluid biomarker levels.

In one embodiment, a prodromal subject treated with the composition comprising pridopidine maintains fluid biomarker levels for a period of 6 months, 1 year, 2 years, 3 years, 4 years, years, 6 years, 7 years, 8 years, 9 years or 10 years. In another embodiment, a subject treated with the composition comprising pridopidine maintains fluid biomarker levels for a period of 10-15 years. In another embodiment, a subject treated with the composition comprising pridopidine maintains fluid biomarker levels for over 15 years.

In one embodiment, a prodromal subject treated with the composition comprising pridopidine demonstrates a reduction in the levels of fluid biomarkers by at least 1%, at least 2%, at least 3%, at least 4%, at least 5%, at least 6%, at least 7%, at least 8%, at least 9% or at least 10%. In another embodiment, a subject treated with the composition comprising pridopidine demonstrates a reduction of 10-20% in fluid biomarker levels. In another embodiment, a prodromal subject treated with the composition comprising pridopidine demonstrates a reduction of 20-30% in fluid biomarker levels. In another embodiment a subject treated with the composition comprising pridopidine demonstrates a reduction of 30-40% in fluid biomarker levels. In another embodiment, a subject treated with the composition comprising pridopidine demonstrates a reduction of 40-50% in fluid biomarker levels.

In some embodiments of the methods disclosed herein, the prodromal subject has at least 36 CAG repeats in the huntingtin gene.

In some embodiments, administering a composition comprising pridopidine to a prodromal HD subject who has at least 36 CAG repeats in the huntingtin (Htt) gene, prevents the clinical onset of HD.

In one embodiment, the clinical onset of HD comprises reduced functional capacity (TFC<13), impairment of motor function (TMS>20 and DCL stage=4), behavioral problems (i.e. depression, anxiety measured by the PBA-S scale or other regulatory accepted clinical scale), personality changes, impairment in gait and balance, cognitive decline (measured by the SDMT, SWR, HD-CAB or other common clinical tests i.e. the mini-mental state examination), involuntary movements, decline in oculomotor function (i.e. antisaccade error rate), decrease in whole brain volume, caudate volume or putamen volume, increase in NfL and IL-6 levels, or any combination thereof.

In one embodiment, the composition for use in the methods of this invention comprises pridopidine or pharmaceutically acceptable salt and Compound 1:

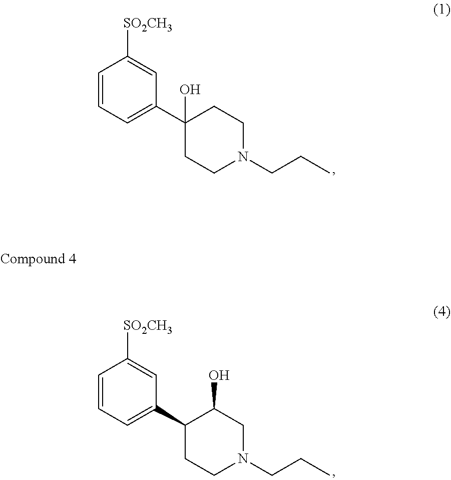 [SMILES: CCCN1CCC(O)(c2cccc(S(C)(=O)=O)c2)CC1 ; CCCN1CC[C@@H](c2cccc(S(C)(=O)=O)c2)[C@@H](O)C1]

combination thereof or pharmaceutically acceptable salts thereof. In another embodiment, the composition comprises pridopidine or pharmaceutically acceptable salt and Compound 1 or pharmaceutically acceptable salt thereof. In another embodiment, the composition comprises pridopidine or pharmaceutically acceptable salt thereof and Compound 4 or pharmaceutically acceptable salts thereof. In another embodiment, the composition comprises pridopidine or pharmaceutically acceptable salt thereof and Compound 1 or pharmaceutically acceptable salt thereof and Compound 4 or pharmaceutically acceptable salt thereof.

In some embodiment, the methods described herein comprise administering pridopidine or a pharmaceutically acceptable salt between 10 mg/day-225 mg/day. In other embodiments pridopidine or pharmaceutically acceptable salt thereof is administered in a daily dose of between 10 mg/day-100 mg/day. In other embodiments pridopidine or pharmaceutically acceptable salt thereof is administered in daily dose of between 10 mg/day-45 mg/day. In other embodiments pridopidine or pharmaceutically acceptable salt thereof is administered in daily dose of between 20 mg/day-60 mg/day. In other embodiments pridopidine or pharmaceutically acceptable salt thereof is administered in daily dose of between 70 mg/day-150 mg/day. In other embodiments pridopidine or pharmaceutically acceptable salt thereof is administered in daily dose of between 45 mg/day-225 mg/day. In other embodiments pridopidine or pharmaceutically acceptable salt thereof is administered in daily dose of between 90 mg/day-225 mg/day.

In one embodiment, the pharmaceutical composition is administered once a day (qd), twice per day (bid) or three times per day. In another embodiment, an equal amount of the pharmaceutical composition is administered at each administration. In an embodiment, the doses are administered at least 6 hours apart, at least 7 hours, at least 8 hours, at least 9 hours, at least 10 hours, at least 11 hours apart.

In one embodiment each of Compound 1, Compound 4 or its pharmaceutically acceptable salt thereof have a weight percentage of 0.001%-10% relative to pridopidine. In one embodiment each of Compound 1, Compound 4 or its pharmaceutically acceptable salt thereof have a weight percentage of 0.001%-1.0% relative to pridopidine. In one embodiment each of Compound 1, Compound 4 or its pharmaceutically acceptable salt thereof have a weight percentage of 0.01%-0.1% relative to pridopidine. In one embodiment each of Compound 1, Compound 4 or its pharmaceutically acceptable salt thereof have a weight percentage of 0.05%-0.2% relative to pridopidine. In one embodiment each of Compound 1, Compound 4 or its pharmaceutically acceptable salt thereof have a weight percentage of 0.05%-0.3% relative to pridopidine. In one embodiment each of Compound 1, Compound 4 or its pharmaceutically acceptable salt thereof have a weight percentage of 0.05%-0.4% relative to pridopidine. In one embodiment each of Compound 1, Compound 4 or its pharmaceutically acceptable salt thereof have a weight percentage of 0.05%-0.5% relative to pridopidine. In one embodiment each of Compound 1, Compound 4 or its pharmaceutically acceptable salt thereof have a weight percentage of 0.1%-0.3% relative to pridopidine. In one embodiment each of Compound 1, Compound 4 or its pharmaceutically acceptable salt thereof have a weight percentage of 0.1%-0.3% relative to pridopidine. In one embodiment each of Compound 1, Compound 4 or its pharmaceutically acceptable salt thereof have a weight percentage of 0.2%-0.5% relative to pridopidine. In another embodiment, each of Compound 1, Compound 4 or its pharmaceutically acceptable salt thereof have a weight percentage of 0.1%-0.9% relative to pridopidine. In another embodiment, each of Compound 1, Compound 4, or its pharmaceutically acceptable salt thereof have a weight percentage of 0.2%-0.8% relative to pridopidine. In another embodiment, each of Compound 1, Compound 4, or its pharmaceutically acceptable salt thereof have a weight percentage of 0.3%-0.7% relative to pridopidine. In another embodiment, each of Compound 1, Compound 4, or its pharmaceutically acceptable salt thereof have a weight percentage of 0.4%-0.6% relative to pridopidine. In another embodiment, each of Compound 1, Compound 4, or its pharmaceutically acceptable salt thereof have a weight percentage of 1%-3% relative to pridopidine. In another embodiment, each of Compound 1, Compound 4, or its pharmaceutically acceptable salt thereof have a weight percentage of 2%-5% relative to pridopidine. In another embodiment, each of Compound 1, Compound 4, or its pharmaceutically acceptable salt thereof have a weight percentage of 4%-7% relative to pridopidine. In another embodiment, each of Compound 1, Compound 4, or its pharmaceutically acceptable salt thereof have a weight percentage of 5%-10% relative to pridopidine.

### Terms

As used herein, and unless stated otherwise, each of the following terms shall have the definition set forth below.

The articles “a”, “an” and “the” are non-limiting. For example, “the method” includes the broadest definition of the meaning of the phrase, which can be more than one method.

“Administering to the subject” or “administering to the (human) patient” means the giving of, dispensing of, or application of medicines, drugs, or remedies to a subject/patient to relieve, cure, or reduce the symptoms associated with a condition, e.g., a pathological condition. The administration can be periodic administration.

As used herein, an “amount” or “dose” of pridopidine as measured in milligrams refers to the milligrams of pridopidine present in a preparation, regardless of the form of the preparation. A “dose of 90 mg pridopidine” means the amount of pridopidine base in a preparation is 90 mg, regardless of the form of the preparation. Thus, when in the form of a salt, e.g. a pridopidine hydrochloride, the weight of the salt form necessary to provide a dose of 90 mg pridopidine would be greater than 90 mg due to the presence of the additional salt ion.

As used herein, “pridopidine” means pridopidine base or a pharmaceutically acceptable salt thereof, as well as derivatives, for example deuterium-enriched version of pridopidine and salts. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference. In certain embodiments, pridopidine is a pharmaceutically acceptable salt, such as the HCl salt or tartrate salt. Preferably, in any embodiments of the invention as described herein, the pridopidine is in the form of its hydrochloride salt.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials.

### Pharmaceutically Acceptable Salts

The active compounds for use according to the invention may be provided in any form suitable for the intended administration. Suitable forms include pharmaceutically (i.e. physiologically) acceptable salts, and pre- or prodrug forms of the compound of the invention.

Examples of pharmaceutically acceptable addition salts include, without limitation, the non-toxic inorganic and organic acid addition salts such as the hydrochloride, the hydrobromide, the L-tartrate, the nitrate, the perchlorate, the phosphate, the sulphate, the formate, the acetate, the aconate, the ascorbate, the benzenesulphonate, the benzoate, the cinnamate, the citrate, the embonate, the enantate, the fumarate, the glutamate, the glycolate, the lactate, the maleate, the malonate, the mandelate, the methanesulphonate, the naphthalene-2-sulphonate, the phthalate, the salicylate, the sorbate, the stearate, the succinate, the tartrate, the toluene-p-sulphonate, and the like. Such salts may be formed by procedures well known and described in the art.

### Pharmaceutical Compositions

While the compounds for use according to the invention may be administered in the form of the raw compound, it is preferred to introduce the active ingredients, optionally in the form of physiologically acceptable salts, in a pharmaceutical composition together with one or more adjuvants, excipients, carriers, buffers, diluents, and/or other customary pharmaceutical auxiliaries.

In an embodiment, the invention provides pharmaceutical compositions comprising the active compounds or pharmaceutically acceptable salts or derivatives thereof, together with one or more pharmaceutically acceptable carriers therefore, and, optionally, other therapeutic and/or prophylactic ingredients know and used in the art. The carrier(s) must be “acceptable” in the sense of being compatible with the other ingredients of the formulation and not harmful to the recipient thereof.

The pharmaceutical composition of the invention may be administered by any convenient route, which suits the desired therapy. Preferred routes of administration include oral administration, in particular in tablet, in capsule, in multiparticulate (beads, granules, mini-tablets) in powder, or in liquid form, and parenteral administration, in particular cutaneous, subcutaneous, intramuscular, or intravenous injection. In some embodiments, the pharmaceutical composition is a solid oral dosage form.

In some embodiment, the pharmaceutical composition is an extended release or modified release formulation comprising at least one pharmaceutically acceptable rate controlling excipient. Non limiting examples of rate controlling excipients include hydrogenated castor oil, polyethylene oxide, ethyl cellulose hydroxypropyl methylcellulose (HPMC), hydroxypropyl cellulose (HPC), polyvinyl alcohol (PVA), vinyl alcohol polymer, polycrylates, polymethacrylates, ethyl acrylate-methyl methacrylate copolymers, glyceryl monostearate, and mixtures thereof. In an embodiment, the total amount of the rate controlling excipients is from about 8% to about 70% of the total weight of the dosage form, from about 10% to about 50% of the total weight of the dosage form, or from about 20% to about 50% of the total weight of the dosage form, from about 30% to about 50% or from about 30% to about 40% of the total weight of the dosage form. In some embodiments, the modified release formulation is as disclosed in WO 2015/112601.

In some embodiment, the pharmaceutical composition is formulated as an immediate release formulation. In some embodiments, the immediate release formulation is as described in WO 2019/046568.

In some embodiments the methods of this invention make use of a pharmaceutical composition comprising pridopidine or pharmaceutically acceptable salt thereof and at least one compound of compounds 1-7 or pharmaceutically acceptable salt thereof, wherein compounds 1-7 are represented by the following structures:

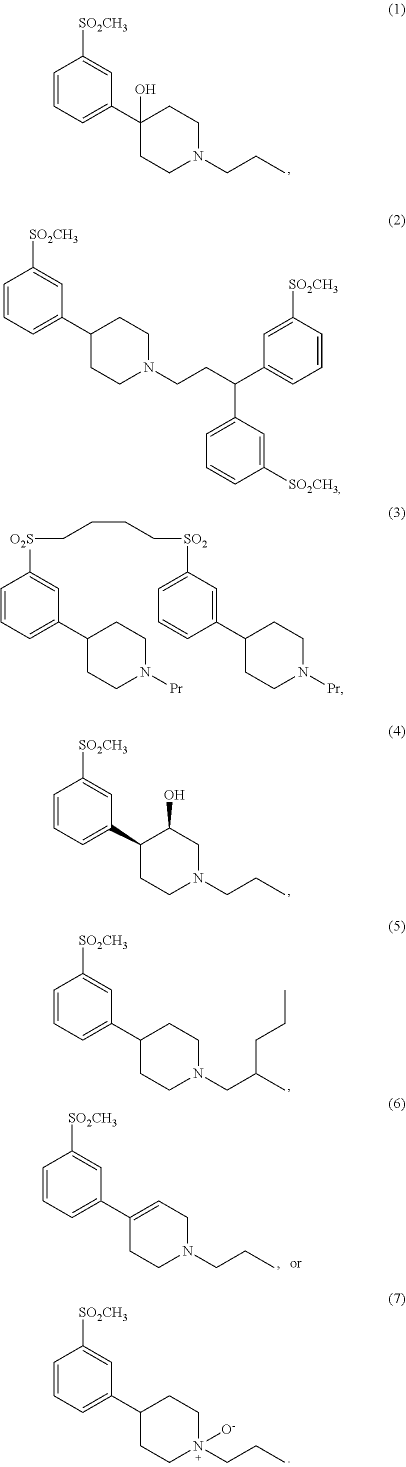

In some embodiments the methods described herein make use of a pharmaceutical composition comprising pridopidine or pharmaceutically acceptable salt thereof and Compound 1 or pharmaceutically acceptable salt thereof. In other embodiments this invention provides a pharmaceutical composition comprising pridopidine or pharmaceutically acceptable salt thereof and Compound 2 or pharmaceutically acceptable salt thereof. In other embodiments this invention provides a pharmaceutical composition comprising pridopidine or pharmaceutically acceptable salt thereof and Compound 3 or pharmaceutically acceptable salt thereof. In other embodiments this invention provides a pharmaceutical composition comprising pridopidine or pharmaceutically acceptable salt thereof and Compound 4 or pharmaceutically acceptable salt thereof. In other embodiments this invention provides a pharmaceutical composition comprising pridopidine or pharmaceutically acceptable salt thereof and Compound 5 or pharmaceutically acceptable salt thereof. In other embodiments this invention provides a pharmaceutical composition comprising pridopidine or pharmaceutically acceptable salt thereof and Compound 6 or pharmaceutically acceptable salt thereof. In other embodiments this invention provides a pharmaceutical composition comprising pridopidine or pharmaceutically acceptable salt thereof and Compound 7 or pharmaceutically acceptable salt thereof. In other embodiments this invention provides a pharmaceutical composition comprising pridopidine or pharmaceutically acceptable salt thereof and Compound 1 and Compound 4 or pharmaceutically acceptable salt thereof.

In one embodiment each of Compound 1, Compound 2, Compound 3, Compound 4, Compound 5, Compound 6, Compound 7 or its pharmaceutically acceptable salt thereof have a weight percentage of 0.001%-10% relative to pridopidine, 0.001%-1.0% w/w relative to pridopidine; 0.005%-0.01% w/w relative to pridopidine; 0.01%-0.1% w/w relative to pridopidine; 0.05%-0.5% w/w relative to pridopidine; 0.05%-0.3% w/w relative to pridopidine; 0.1%-1% w/w relative to pridopidine; 1-5% w/w relative to pridopidine; 1-10% w/w relative to pridopidine; 5-10% w/w relative to pridopidine.

In some embodiments the methods of this invention make use of a pharmaceutical composition comprising pridopidine or pharmaceutically acceptable salt thereof and at least one compound of compounds 1-7 or pharmaceutically acceptable salt thereof; wherein pridopidine and/or compounds 1-7 or salts thereof are deuterium-enriched. Examples of deuterium-enriched pridopidine and salts and their methods of preparation may be found in U.S. Application Publication Nos. 2013-0197031, 2016-0166559 and 2016-0095847, the entire content of each of which is hereby incorporated by reference.

“Deuterium-enriched” means that the abundance of deuterium at any relevant site of the compound is more than the abundance of deuterium naturally occurring at that site in an amount of the compound. The naturally occurring distribution of deuterium is about 0.0156%. Thus, in a “deuterium-enriched” compound, the abundance of deuterium at any of its relevant sites is more than 0.0156% and can range from more than 0.0156% to 100%. Deuterium-enriched compounds may be obtained by exchanging hydrogen with deuterium or synthesizing the compound with deuterium-enriched starting materials.

Further details on techniques for formulation and administration may be found in the latest edition of Remington's Pharmaceutical Sciences (Mack Publishing Co., Easton, PA).

### Examples

### Example 1: Treatment of Prodromal HD with Pridopidine

The efficacy of pridopidine for treating prodromal HD is evaluated in a randomized, double-blind, placebo-controlled trial. Participants are randomized 1:1 to receive either pridopidine 45 mg bid (n=50) or placebo (n=50) for a period of 52 weeks. Inclusion criteria are ≥36 CAG repeats in the HTT gene, DCL of 2 or 3, UHDRS-TMS of 5-10, TFC of 13, Symbol Digits Modalities Test (SDMT) score of 40-60 and a CAP score [age×(CAG-33.66)]≥ 250.

The efficacy of pridopidine is assessed by motor assessments, structural and functional imaging, metabolic imaging (FDG-PET), fluid biomarker levels and cognitive and psychiatric assessments.

Pridopidine 45 mg bid demonstrates a maintenance, improvement or less decline compared to placebo in motor measures TMS and Q-Motor finger tapping. Pridopidine-treated patients demonstrate no decrease or less decrease compared to placebo in the volume of the striatal structures caudate and putamen, as well as total brain volume and cortical thickness.

Pridopidine treated patients also show a decrease or maintenance of ventricular volume or a smaller increase in ventricular volume compared to placebo.

Participants receiving pridopidine 45 mg bid show maintenance of or less worsening in DMN and functional connectivity compared to placebo-treated participants. Pridopidine 45 mg bid improves or maintains or show less worsening compared to placebo in cognitive assessments as assessed by the HD-CAB battery of tests. Pridopidine 45 mg bid improves, maintains or shows less decline in cognitive assessments compared to placebo as assessed by the SDMT. Pridopidine treatment demonstrates an improvement or less worsening compared to placebo in the PBA-s total score and the PBA-s apathy score.

Additionally, pridopidine demonstrates reduction or maintenance of the levels of fluid biomarkers NfL, phospho-tau and proenkephalin in CSF and/or plasma vs. placebo.

Over the course of the trial, less participants in the pridopidine group progressed to manifest HD compared to the placebo group.

These data demonstrate that pridopidine 45 mg bid is efficacious for treating prodromal HD and delaying the onset of symptoms leading to the diagnosis of manifest HD.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1. A method of treating prodromal Huntington Disease (HD) in a subject who has at least 36 CAG repeats in the huntingtin (Htt) gene, wherein said method comprises administering a composition comprising pridopidine or a pharmaceutically acceptable salt thereof.

2. The method of claim 1, wherein said prodromal HD subject has Unified Huntington's Disease Rating Scale-Total Functional Capacity (UHDRS-TFC) of 13.

3. The method of claim 1, wherein said prodromal HD subject has a Diagnostic Confidence Level (DCL) of 1, 2 or 3.

4. The method of claim 1, wherein said prodromal HD subject is characterized by impairment of motor functions comprising Total Motor Scale (TMS) with a score of between 5 and 10.

5. The method of claim 1, wherein said prodromal HD subject has an IS ≥90%.

6. The method of claim 1, wherein said prodromal HD subject is in Stage 1 or Stage 2 in the HD Integrated Staging System (HD-ISS).

7. The method of claim 1, wherein said prodromal HD subject has a Symbol Digits Modality Score (SDMT) of between 40 and 60.

8. The method of claim 1, wherein the method comprises administering pridopidine or a pharmaceutically acceptable salt between 10 mg/day-225 mg/day.

9. The method of claim 1, wherein the composition comprises additional Compound 1: 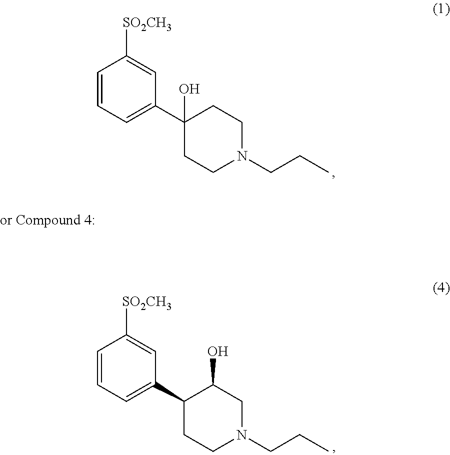 [SMILES: CCCN1CCC(O)(c2cccc(S(C)(=O)=O)c2)CC1 ; CCCN1CC[C@@H](c2cccc(S(C)(=O)=O)c2)[C@@H](O)C1]
or combination thereof; or pharmaceutically acceptable salts thereof.

10. The method of claim 9, wherein Compound 1 or Compound 4 have a weight percentage of 0.001%-1.0% relative to pridopidine.

11. The method of claim 10, wherein Compound 1 or Compound 4 have a weight percentage of 0.05%-0.5% relative to pridopidine.

12. The method of claim 9, wherein Compound 1 or Compound 4 have a weight percentage of 0.05%-0.3% relative to pridopidine.

13. The method of claim 1, wherein the pharmaceutically acceptable salt is hydrochloride, hydrobromide, hydroiodide, nitrate, sulfate, bisulfate, phosphate, acid phosphate, isonicotinate, acetate, lactate, salicylate, citrate, D,L-tartrate, L-tartarate, D-tartarate, pantothenate, bitartrate, ascorbate, succinate, hemisuccinate, maleate, gentisinate, gentisate, fumarate, gluconate, glucaronate, glycolate, saccharate, formate, besylate, benzoate, glutamate, malate, methanesulfonate, ethanesulfonate, benzensulfonate, p-toluenesulfonate, oxalate, tosylate, naphtalen-2-sulfate, pamoate (i.e., 1,1′-methylene-bis-(2-hydroxy-3-naphthoate)), perchlorateaconate,cinnamate's citrate, embonate, enantate, malonate, mandelate, phthalate, sorbate or stearate salt.

14. The method of claim 9, wherein the pharmaceutically acceptable salt is hydrochloride, hydrobromide, hydroiodide, nitrate, sulfate, bisulfate, phosphate, acid phosphate, isonicotinate, acetate, lactate, salicylate, citrate, D,L-tartrate, L-tartarate, D-tartarate, pantothenate, bitartrate, ascorbate, succinate, hemisuccinate, maleate, gentisinate, gentisate, fumarate, gluconate, glucaronate, glycolate, saccharate, formate, besylate, benzoate, glutamate, malate, methanesulfonate, ethanesulfonate, benzensulfonate, p-toluenesulfonate, oxalate, tosylate, naphtalen-2-sulfate, pamoate (i.e., 1,1′-methylene-bis-(2-hydroxy-3-naphthoate)), perchlorateaconate, cinnamate's citrate, embonate, enantate, malonate, mandelate, phthalate, sorbate or stearate salt.

15. The method of claim 2, wherein said prodromal HD subject is characterized by impairment of motor functions comprising Total Motor Scale (TMS) with a score of between 5 and 10.
